(19) United States
(12) Patent Application Publication
(10) Pub. No.: US 2008/0241871 A1
(43) Pub. Date: Oct. 2, 2008

(54) Luminescence measuring apparatus

(75) Inventors: Masahiro Okanojo, Hachioji (JP); Satoshi Ozawa, Musashino (JP); Hideyuki Noda, Kokubunji (JP); Masaaki Hirano, Tokyo (JP)

Correspondence Address: Stanley P. Fisher;Reed Smith LLP, 3110 Fairview Park Drive, Suite 1400, Falls Church, VA, 22042-4503, US

(73) Assignee: Hitachi Plant Technologies, Ltd.

(21) Appl. No.: 12/010,497

(22) Filed: Jan. 25, 2008

(30) Foreign Application Priority Data
Mar. 30, 2007 (JP) 2007-093955

(51) Int. Cl. C12Q 1/02; G01N 21/76; C12M 1/00

(52) U.S. Cl. 435/29; 422/52; 435/288.7; 436/172

(57) ABSTRACT

The present invention provides a luminescence measuring method that can be accurately and quickly carried out while inhibiting a possible background associated with viable bacteria adhering to a nozzle or Adenosine Tri Phosphate remaining in the nozzle, and an apparatus for the method. The present invention uses a washing apparatus characterized by including a nozzle, a lysys solution, a luminescence reagent solution, and a detection section, as well as a relevant washing method and a relevant luminescence measuring method. To remove viable bacteria adhering to the nozzle, the nozzle is immersed in the lysys solution and then in the luminescence reagent solution. The detection section monitors luminescence occurring during a washing process.

### CLAIM OF PRIORITY

The present application claims priority from Japanese application JP 2007-093955 filed on Mar. 30, 2007, the content of which is hereby incorporated by reference into this application.

### BACKGROUND OF THE INVENTION

1. Field of the Invention

The present invention relates to a luminescence measuring apparatus that can be accurately and easily operated at a high speed, and a luminescence measuring method that can be carried out accurately, quickly, and easily. More specifically, the present invention relates to a biochemical luminescence measuring apparatus that can accurately measure the number of viable bacteria in an infinitesimal quantity at a 1 CFU level, as well as a relevant luminescence measuring method.

2. Background Art

In environments such as various clinical medicine sites, food factories, and basic research sites which require sterility and biological cleanliness, the number of microorganisms (microbial count) in the air is generally measured. In particular, in an environment such as the drug industry, a treatment reference value for environment monitoring for the microbial count is 1 CFU (Colony Forming Unit; the unit of the microbial count)/m³ for airborne bacteria, 1 CFU/24 to 30 cm² for surface adherent bacteria (equipment), 1 CFU/24 to 30 cm² for surface adherent bacteria (walls), 5 CFU/24 to 30 cm² for surface adherent bacteria (floors), 1 CFU/5 fingers for finger adherent bacteria, and 5 CFU/24 to 30 cm² for workwear surface adherent bacteria. That is, the treatment reference value is at most 5 CFU in all the cases. If this value is exceeded, it is necessary to immediately conduct investigations and take corrective measures as required.

A method described in Japanese Pharmacopoeia has hitherto been used to measure the number of microorganisms in a sample; the method uses a sample intact or filters viable bacteria from the sample to culture the viable bacteria in a standard agar medium for a long time and detects a colony in the medium. However, this method has the disadvantage of requiring a long time.

As another method of measuring the microbial count, a biochemical luminescence measuring method utilizing a luminescence reaction of enzyme luciferase, substrate luciferin Adenosine Tri Phosphate is often used because of the speed and easiness of the method. First, Adenosine Tri Phosphate is extracted from viable bacteria to be measured, and decomposed for luminescence using a luminescence reagent containing luciferase and luciferin and catalyzing a luminescence reaction using Adenosine Tri Phosphate as a substrate. The quantity of Adenosine Tri Phosphate is determined from the quantity of luminescence. The microbial count is calculated from the determined Adenosine Tri Phosphate quantity and the quantity of Adenosine Tri Phosphate per viable bacterium. An example of a method of measuring the microbial count utilizing the above-described luminescence measuring method is described in a thesis report published by Kikkoman Corporation. (Analytical Biochemistry, 2003, 319, p 287 to 295). This report shows that the lower limit on the detection of Escherichia coli (E. coli) is about 10 CFU on the basis of a calibration curve for determination of the number of E. coli bacteria. That is, the luminescence measuring method has an insufficient sensitivity for the environment monitoring applications.

A luminescence measuring apparatus capable of automatic measurements or repeated measurements dispenses a sample solution containing a target in a reaction container, binds the target to a fluorescent reagent in the reaction container, irradiates the bind with excitation light, automatically measures the resultant fluorescence, and repeats the measurement. A dispensing nozzle is commonly used for dispensing operations because of the convenience of the nozzle for automatic or repeated measurements. However, with the dispensation with the nozzle, the target may remain in the nozzle, affecting measurement accuracy or sensitivity. In contrast, a known technique attempts to improve the accuracy and sensitivity of the measurement by repeatedly sucking and ejecting a cleaning fluid into and from the nozzle for each measurement to wash the nozzle (JP Patent Publication (Kokai) No. 9-304243). However, with this technique, the nozzle washing only achieves dilution with the cleaning fluid and fails to confirm that the target remaining in the nozzle has been successfully removed.

The present inventor attempted to apply the luminescence measuring method and washing method according to the conventional art to automatic and repeated measurements so as to very sensitively measure the number of viable bacteria in an infinitesimal quantity of at most 5 CFU and at a 1 CFU level. Then, the present inventor newly found the following problems.

Since Adenosine Tri Phosphate is contained in the cells in all organisms, Adenosine Tri Phosphate exists not only in viable bacteria but also in single-cell and multicellular organisms. Furthermore, free Adenosine Tri Phosphate exists around these cells. If Adenosine Tri Phosphate from a source in a measurement environment which source is different from the viable bacteria to be measured adheres to the nozzle or alternate measurements are made of a sample solution containing a small quantity of Adenosine Tri Phosphate and a sample solution containing a large quantity of Adenosine Tri Phosphate, viable bacteria other than those to be measured may mix into the sample solution or Adenosine Tri Phosphate from another sample may remain in the nozzle. Thus, the remaining Adenosine Tri Phosphate may be measured as a variation in background luminescence. The measurement sensitivity and measurement accuracy in this case are insufficient to measure the number of viable bacteria in an infinitesimal quantity. Furthermore, since the conventional washing method only carries out dilution with the cleaning fluid, it is difficult for the method to remove viable bacteria adhering to the nozzle or remaining Adenosine Tri Phosphate. It is thus difficult for the conventional washing method to sensitively achieve automatic and repeated measurements at the 1 CFU level. Furthermore, owing to the lack of appropriate means for checking the washing step, the washing step may be insufficient, reducing the sensitivity or accuracy, or reducing the time required for the washing step may be difficult. Consequently, the microbial count cannot be quickly measured.

An object of the present invention is to provide a luminescence measuring method that can be accurately and quickly carried out while inhibiting the possible background of viable bacteria adhering to the nozzle or remaining Adenosine Tri Phosphate, and an apparatus for the method.

### SUMMARY OF THE INVENTION

As a result of examinations for solving the above-described problems, the present inventor has found that accurate luminescence measurements can be achieved by washing the nozzle with a luminescence reagent to in advance remove microorganisms which adhere to the nozzle but which are not measurement targets as well as remaining Adenosine Tri Phosphate and then using the cleaned nozzle to dispense a sample solution in a reaction container. The present inventor has thus completed the present invention.

The present invention relates to a luminescence measuring apparatus comprising a sample container accommodating a sample solution, a luminescence reagent vessel accommodating a luminescence reagent solution for detection of the sample solution, a reaction container in which the sample solution and the luminescence reagent solution are allowed to react chemically, a detection section detecting luminescence in the reaction container, a nozzle sucking and ejecting the sample solution, an arm section controlling operation of the nozzle, and a pressure control section controlling pressure on the nozzle, wherein before and after a measuring step of ejecting the sample solution sucked into the nozzle, into the reaction container for luminescence detection, the arm section introduces the nozzle into the reaction container filled with the luminescence reagent solution in order to decompose and wash out a luminescence substrate remaining on a surface of the nozzle.

The luminescence measuring apparatus may further comprise a lysys container accommodating a lysys solution, and the arm section may introduce the nozzle into the lysys solution in order to lyse viable bacteria adhering to the nozzle surface.

To increase the accuracy of suction into the nozzle, the pressure control section preferably controls pressure before and after the sample solution is sucked into the nozzle so that gas or a liquid not mixing with the sample solution is sucked into the nozzle.

The luminescence measuring apparatus may further comprise a storage section storing a detection value detected by the detection section and a comparative calculation section comparing the detection value stored in the storage section with a threshold value.

The luminescence measuring apparatus may further comprise a control section switching between the washing step and the measurement step on the basis of a result of comparison of a threshold value with a detection value for a background detected during the washing step and/or lysys step and stored in the storage section, the comparison being carried out by the comparative calculation section.

The present invention also relates to a luminescence measuring method comprising executing, before and after a measurement step of sucking a sample into a nozzle, ejecting the sample solution through the nozzle into a reaction container filled with a luminescence reagent solution, and detecting luminescence in the reaction container, a washing step of immersing the nozzle into which the sample solution has been sucked, in the reaction container filled with the luminescence reagent solution and decomposing a luminescence substrate (Adenosine Tri Phosphate) remaining on a surface of the nozzle.

The method may further comprise a step of, before the washing step, immersing the nozzle into which the sample solution has been sucked, in a lysys solution to lyse viable bacteria adhering to the nozzle surface.

The method may further comprise a step of monitoring luminescence detected during the washing step and/or lysys step.

The method may further comprise a step of, before and after sucking the sample solution into the nozzle, sucking gas or a liquid not mixing with the sample solution into the nozzle to increase the accuracy of suction into the nozzle.

In the method, a detection value for a background detected during the washing step and/or lysys step may be compared with a threshold value, and on the basis of a result of the comparison, the washing step and the measurement step may be switched.

The present invention carries out, before and after the measurement step, the washing step of using the luminescence reagent solution or the lysys solution to remove the viable bacteria which adhere to the nozzle but which are different from measurement targets as well as remaining Adenosine Tri Phosphate. Thus, the number of viable bacteria in an infinitesimal quantity at the 1 CFU level can be automatically measured very sensitively. The present invention can also use the detection section to monitor luminescence occurring during the washing step to check whether or not the quantity of the viable bacteria adhering to the nozzle or the remaining Adenosine Tri Phosphate has decreased to a predetermined level or less. Thus, the time required for the washing step can be reduced to optimize the washing step. Moreover, the present invention can quickly and easily measure the number of viable bacteria in an infinitesimal quantity and thus monitor the number of viable bacteria in an infinitesimal quantity in real time.

### BRIEF DESCRIPTION OF THE DRAWINGS

FIG. 1 is a diagram showing an embodiment of a washing apparatus according to the present invention;

FIG. 2 is a diagram showing the results of washing of a nozzle with a cleaning fluid and a lysys solution;

FIG. 3 is a diagram showing the results of washing of a nozzle with the cleaning fluid and a luminescence reagent solution;

FIG. 4 is a diagram illustrating the flow of a process carried out by the washing apparatus according to the present invention;

FIG. 5 is a diagram illustrating a process of washing the nozzle and a process of measuring the reagent solution;

FIG. 6 is a diagram showing the results of a series of luminescence measurements during the washing and measurement processes;

FIG. 7 is a diagram showing the results of luminescence measurements for 1, 2, 5, 10, 50, 100, and 1,000 atto mol (10⁻¹⁸ mol) Adenosine Tri Phosphate using the washing apparatus, washing method, and luminescence measuring method according to the present invention; and

FIG. 8 is a diagram showing the results of luminescence measurements for 1, 2, 5, 10, 20, and 45 CFU using the washing apparatus, washing method, and luminescence measuring method according to the present invention.

### DETAILED DESCRIPTION OF THE PREFERRED EMBODIMENTS

The present invention will be described below through examples with reference to the drawings. However, the present invention is not limited to the examples.

### EXAMPLE 1

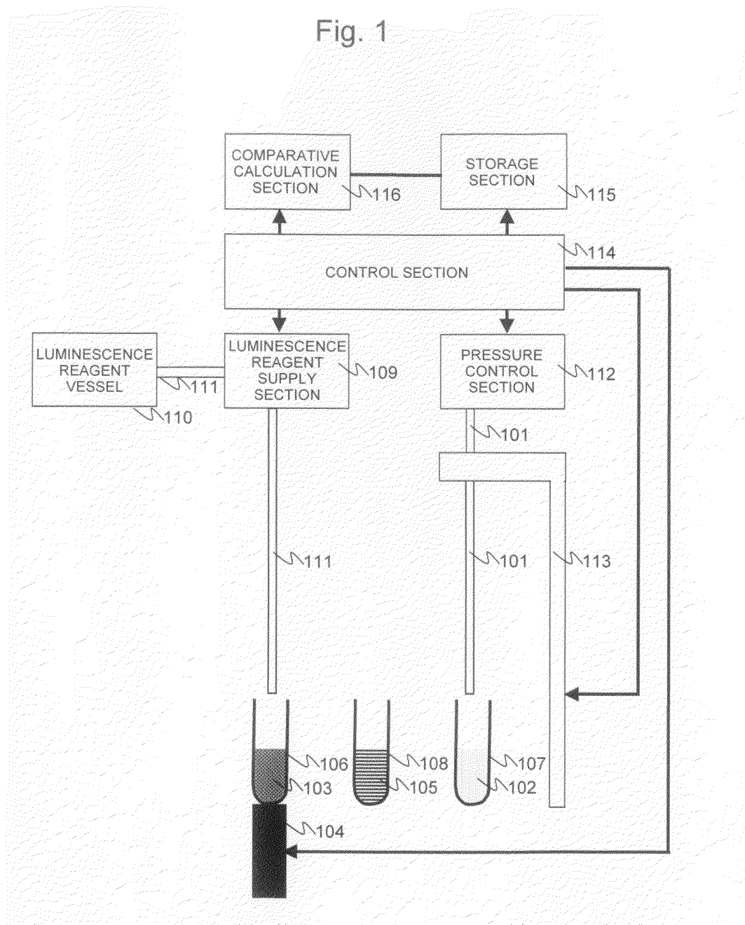

The configuration of a washing apparatus based on Example 1 will be described with reference to FIG. 1.

The washing apparatus is composed of a nozzle 101, a lysys solution 102, a luminescence reagent solution 103, a detection section 104, a sample solution 105, a reaction container 106, a lysys container 107, a sample container 108, a luminescence reagent supply section 109, a luminescence reagent vessel 110, piping 111, a pressure control section 112, an arm section 113, a control section 114, a storage section 115, and a comparative calculation section 116.

To examine the removal of viable bacteria adhering to the nozzle, first, the nozzle was washed using, for example, pure water for the conventional technique instead of the lysys solution 102, a component of the present invention (this is hereinafter referred to as washing A). Subsequently, for the present invention, the nozzle was washed using the lysys solution 102, a component of the present invention (this is hereinafter referred to as washing B). The results of the conventional technique were thus compared with the results of the present invention.

In Example 1, a silica capillary tube manufactured by GL Science Inc. was used as the nozzle 101. Not only glass but also resin or metal may be used as a material for the nozzle. Instead of the capillary, a tip or a resin tube used for a pipette or the like may be used as the nozzle.

The lysys solution 102 was an ATP extract reagent contained in a lucifer HS set manufactured by Kikkoman Corporation and which is effective for lysing viable bacteria. It is also possible to use a solution composed one or more of a surfactant such as benzalkonium chloride, Triton X-100, Triton X-114, Nonidet P-40, Tween 20, Tween 80, Brij-35, Brij-58, 3-[3-cholamidopropyl]-dimethylammonio]-1-propane-sulfonate n-Octyl-s-glucoside, or n-Octyl-s-D-thioglucoside, a protein denaturant such as trichloroacetic acid, sodium dodecyl sulfate, guanidine hydrochloride, or urea, a lysys enzyme such as lysozyme, an organic solvent such as alcohol, ether, or phenol, a strong alkali solution of at least pH 10, and a strong acid solution of at most pH 4.

A luminescence reagent HS contained in the lucifer HS set manufactured by Kikkoman Corporation was used as the luminescence reagent solution 103. The luminescence reagent solution may be an enzyme that is effective for catalyzing a chemical reaction in which a luminescence substance emits light, for example, a solution containing luciferase, peoxidase, or alkaline phosphatase.

In the washing A, corresponding to the conventional technique, the nozzle 101 was immersed first in pure water and then in the luminescence reagent solution 103 in the reaction container 106. The nozzle washing process was monitored by the detection section 104 based on a photon counting scheme.

In the washing B, corresponding to the present invention, the nozzle 101 was immersed in the lysys solution 102 in the lysys container 107 and then in the luminescence reagent solution 103 in the reaction container 106. The nozzle washing process was monitored by the detection section 104.

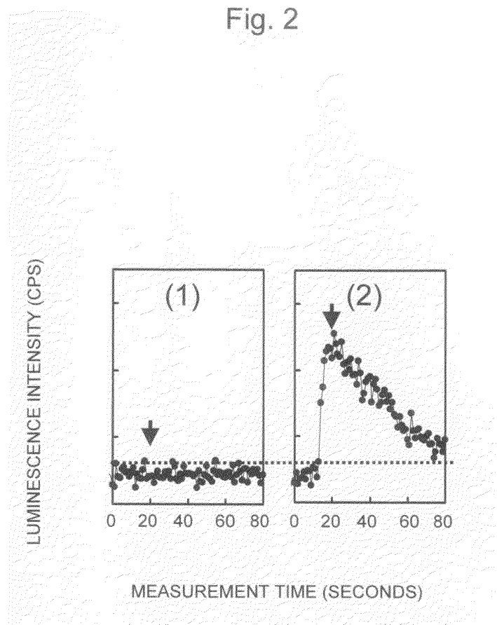

FIG. 2 shows the results of the monitoring of the nozzle 101 washing process carried out by the detection section 104. In FIG. 2, the axis of ordinate indicates luminescence intensity, and the axis of abscissa indicates measurement time. The luminescence intensity is detected as a count per second (CPS) in accordance with the photon counting scheme. A dashed line indicates the average value for a background in the luminescence reagent solution 103 as a threshold value. Arrows in FIGS. 2(1) and 2(2) show the point in time when the nozzle 101 was immersed in the luminescence reagent solution 103.

FIG. 2(1) shows the results of the washing A, corresponding to the conventional technique. FIG. 2(1) shows that no luminescence occurs even when the nozzle 101 is immersed in the luminescence reagent solution 103.

FIG. 2(2) shows the results of the washing B, corresponding to the present invention. FIG. 2(2) shows that luminescence occurs when the nozzle 101 is immersed in the luminescence reagent solution 103.

As shown in FIG. 2, no luminescence was observed during the washing A, corresponding to the conventional technique. It is expected that at least Adenosine Tri Phosphate does not adhere to the nozzle 101 or that even though Adenosine Tri Phosphate adheres to the nozzle 101, the Adenosine Tri Phosphate is accommodated inside the cell membranes of viable bacteria adhering to the nozzle and does not leak out from the bacteria as a result of the washing method A. Subsequently, luminescence was observed during the washing B, corresponding to the present invention. This indicates that immersing the nozzle 101 in the lysys solution 102 allowed viable bacteria adhering to the nozzle to be lysed and that immersing the nozzle 101 in the luminescence reagent solution 103 allowed the Adenosine Tri Phosphate from viable bacteria remaining in the nozzle 101 to be decomposed for luminescence by the luminescence reagent solution 103. The level of the luminescence resulting from the decomposition of Adenosine Tri Phosphate decreased to the level of the luminescence of the background in the luminescence reagent solution 103. This indicates that even when Adenosine Tri Phosphate or viable bacteria remained in the nozzle, the Adenosine Tri Phosphate remaining in the nozzle was decomposed so that the level of the luminescence is equivalent to that of the luminescence of the background in the luminescence reagent solution 103 to wash the nozzle. The results also indicate that the present invention enables the process of removing Adenosine Tri Phosphate from the viable bacteria remaining in the nozzle 101 to be monitored by using the detection section 104 to detect luminescence resulting from the decomposition of the Adenosine Tri Phosphate under the effect of the luminescence reagent solution.

In Example 1, the background in the luminescence reagent solution 103 is used as a threshold value. However, if the background in the luminescence reagent solution 103 is equivalent to a dark count in the detection section 104, the dark count in the detection section 104 can be used as a threshold value.

As described above, the washing B in the present example enables the viable bacteria adhering to the nozzle 101 to be removed and that the nozzle can be washed so that the level of the luminescence decreases to that of the luminescence of the background in the luminescence reagent solution 103. The apparatus according to the present invention comprises the detection section 104, which can monitor the nozzle 101 washing process. By detecting luminescence resulting from the decomposition of Adenosine Tri Phosphate, the apparatus can immediately stop the washing once the luminescence disappears, that is, the decomposition of the Adenosine Tri Phosphate ends. As a result, the time required for the washing process can be reduced.

### EXAMPLE 2

In Example 2, examinations were made of the effects of the removal of Adenosine Tri Phosphate remaining in the opening of the nozzle 101 or on a wall of the nozzle when the nozzle dispenses the sample solution 105. In Example 2, the ATP solution was used as the sample solution 105. The sample solution may be a substance that emits light when subjected to a chemical reaction by an enzyme, such as luciferin, orthophosphoric monoester, or a peroxidase substrate.

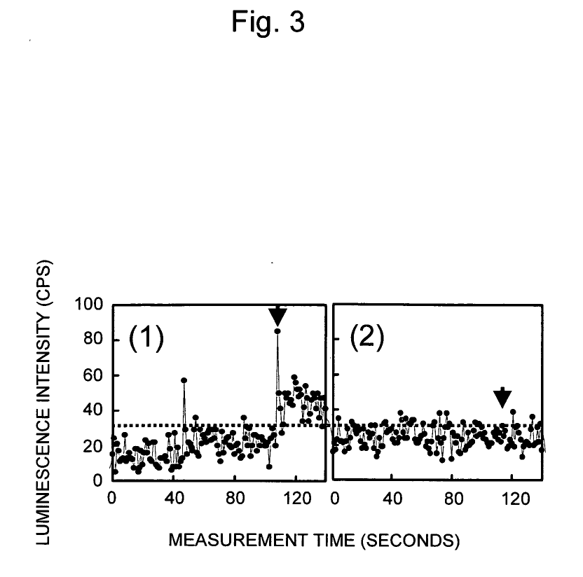

Using the washing apparatus comprising the nozzle 101, the luminescence reagent solution 103, and the detection section 104, as well as the washing method and the luminescence measuring method, which are the characteristics of the present invention, the nozzle 101 was immersed first in the sample solution 105 in the sample container 108 and then in the luminescence reagent solution 103 in the reaction container 106. Adenosine Tri Phosphate from the sample solution 105 adhering to the nozzle 101 was decomposed. The nozzle washing process was monitored by the detection section 104. The results of the monitoring are shown in FIG. 3(1). Subsequently, the nozzle 101 was removed from the luminescence reagent solution 103. To check whether or not Adenosine Tri Phosphate remained in the nozzle 101, the nozzle 101 was immersed in the luminescence reagent solution 103 again and was monitored by the detection section 104. The results of the monitoring are shown in FIG. 3(2).

Arrows in the FIG. 3 indicate the point in time when the nozzle 101 was immersed in the luminescence reagent solution 103. Dashed lines in FIG. 3 indicate the average value (threshold value) for a background in the luminescence reagent solution 103.

As shown in FIG. 3(1), luminescence was observed when the nozzle 101 was immersed in the luminescence reagent solution 103. This indicates that immersing the nozzle 101 in the luminescence reagent solution 103 allowed Adenosine Tri Phosphate from the sample solution 105 remaining in the nozzle 101 to be decomposed for luminescence. The level of the luminescence resulting from the decomposition of the Adenosine Tri Phosphate decreased to that of the luminescence of the background in the luminescence reagent solution 103. This indicates that the Adenosine Tri Phosphate remaining in the nozzle was decomposed so that the level of the luminescence of the Adenosine Tri Phosphate was equivalent to that of the luminescence of the background in the luminescence reagent solution 103 to wash the nozzle.

FIG. 3(2) shows the results of monitoring, by the detection section 104, of the nozzle 101 in FIG. 3(1) removed from the luminescence reagent solution 103 and then immersed in the luminescence reagent solution 103 again. FIG. 3(2) shows that no luminescence occurred when the nozzle 101 was immersed in the luminescence reagent solution 103 again, indicating no Adenosine Tri Phosphate remaining in the nozzle 101.

As described above, the luminescence reagent solution 103 allows the Adenosine Tri Phosphate remaining the nozzle 101 to be removed so that the level of the luminescence of the Adenosine Tri Phosphate is equivalent to that of the luminescence of the background. Furthermore, the detection section 104 can monitor the process of removing the Adenosine Tri Phosphate remaining in the nozzle 101. Additionally, the apparatus according to the present invention, comprising the detection section 104, can monitor the nozzle 101 washing process. Moreover, detecting the luminescence resulting from the decomposition of the Adenosine Tri Phosphate makes it possible to immediately end the washing once the luminescence disappears, that is, once the decomposition of the Adenosine Tri Phosphate is finished. This enables a reduction in the time required for the washing process.

### EXAMPLE 3

In Example 3, Adenosine Tri Phosphate and viable bacteria were quantitatively measured. An ATP solution or a bacteria suspension with viable bacteria lysed therein was used as the sample solution 105. The number of moles in the Adenosine Tri Phosphate added to the luminescence reagent solution 103 was set at 1, 2, 5, 10, 50, 100, and 1,000 atto mol. The microbial count was set at 1, 2, 5, 10, 20, and 45 CFU. The viable bacteria may be GRam-negative bacteria such as pseudomonas aeruginosa or serratia marcescens, Gram-positive bacteria such as basillus subtilis, micrococcus luteus, or staphylococcus aureus, or yeasts or fungi such as candida albicans, cryptococcus albidus, or saccharomyces cerevisiae. Since the viable bacteria are living cells and Adenosine Tri Phosphate is contained in all the cells, animal cells or plant cells that are living cells may be used.

The process in Example 3 will be described below. First, the viable bacteria adhering to the nozzle 101 were removed using the lysys solution 102 and the luminescence reagent solution 103. Then, the sample solution 105 was dispensed in the container. The sample solution 105 remaining in the nozzle 101 was removed using the luminescence reagent solution 103. The sample solution 105 dispensed by the nozzle was added to the luminescence reagent solution 103. Finally, Adenosine Tri Phosphate remaining in the nozzle 101 after the dispensation was removed by sucking and ejecting the luminescence reagent solution 103.

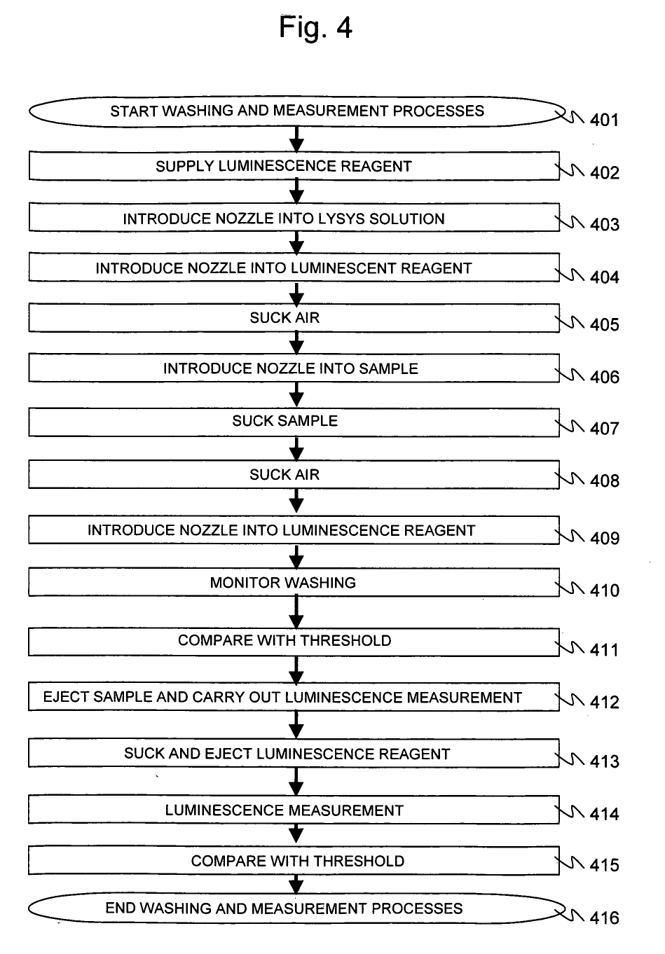

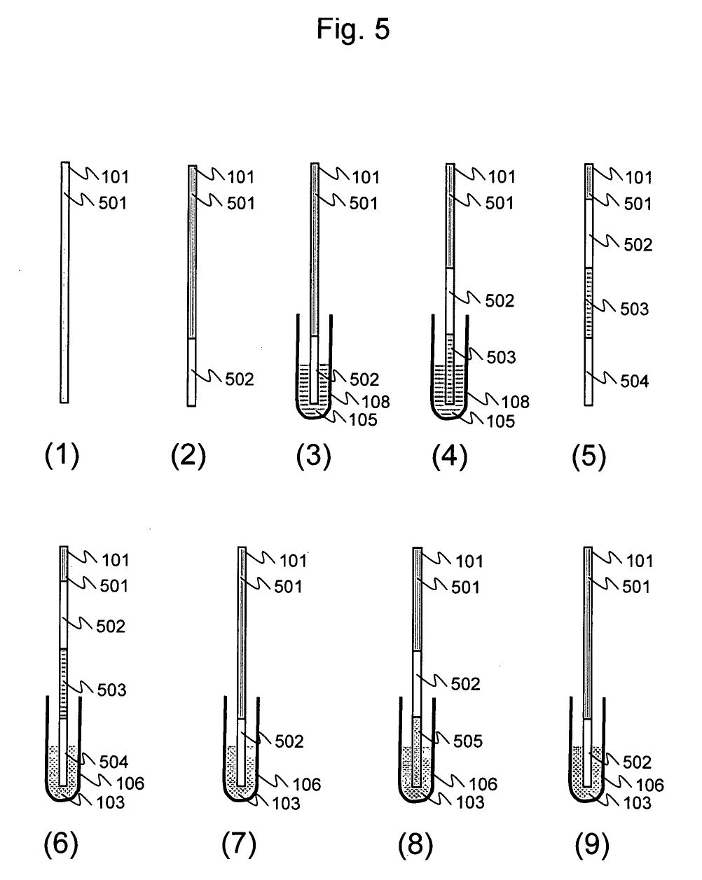

The washing and measurement processes are carried out in accordance with the flow shown in FIG. 4. To increase the dispensation accuracy of the nozzle 101, the nozzle 101 was filled with a buffer 501 as shown in FIG. 5(1). A solution or an organic solvent containing water or a surfactant may be used in place of the buffer. When the buffer contains an enzyme such as apyrase, deaminase, or luciferase which catalyzes the decomposition of Adenosine Tri Phosphate or a chemical substance decomposing or modifying Adenosine Tri Phosphate, Adenosine Tri Phosphate is prevented from mixing into the buffer in the nozzle. Washing can be achieved by discharging through the nozzle the buffer, the buffer containing the enzyme catalyzing the decomposition of Adenosine Tri Phosphate, or a solution, water, or an organic solvent containing a surfactant. In this case, the Adenosine Tri Phosphate is the measurement target. If another target is measured, measurements can be similarly achieved by adding a component that decomposes the measurement target, to the buffer on the basis of the idea of the present invention.

The washing and measurement processes were started (step 401). The luminescence reagent supply section 109 fed the luminescence reagent solution 103 from the luminescence reagent vessel 110 to the reaction container 106 through the piping 111 (step 402). The arm section 113 immerses the nozzle 101 in the lysys solution 102 (step 403) and then in the luminescence reagent solution 103 (step 404). The pressure control section 112 sucked air 502 into the nozzle 101 as shown in FIG. 5(2) (step 405). The arm section 113 immersed the nozzle 101 in the sample solution 105 as shown in FIG. 5(3) (step 406). The pressure control section 112 sucked a sample solution 503 into the nozzle 101 as shown in FIG. 5(4) (step 407). Air 504 was then sucked into the nozzle 101 to sandwich the sample solution 503 between the air layers as shown in FIG. 5(5) (step 408). The arm section 113 immersed the nozzle 101 in the luminescence reagent solution 103 as shown in FIG. 5(6) (step 409). The resultant luminescence was monitored by the detection section 104 (step 410).

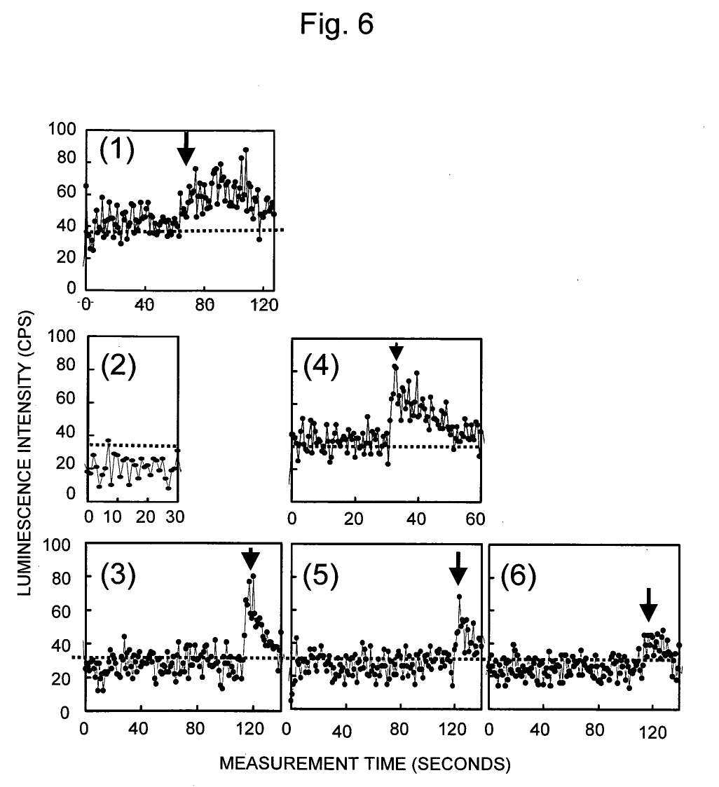

FIG. 6 shows measurement results obtained by the procedure of the washing and measurement processes in FIG. 4. Dashed lines in FIG. 6 show the average value (threshold value) for a background in the luminescence reagent solution 103.

FIG. 6(1) shows the results of monitoring, by the detection section 104, of the nozzle 101 washing process carried out with the sample solution 503 held in the nozzle 101. An arrow in FIG. 6(1) shows the point in time when the nozzle 101 was immersed in the luminescence reagent solution 103. FIG. 6(1) indicates that immersing the nozzle 101 in the luminescence reagent solution 103 increased luminescence intensity above the threshold value shown by the dashed line. The increased luminescence intensity decreased over time. FIG. 6(1) indicates that Adenosine Tri Phosphate remaining in the nozzle 101 caused luminescence while being decomposed.

The control section 114 stores the monitoring value in the storage section 115 as a detection value. The comparative calculation section 116 compared the detection value stored in the storage section 115 with the threshold value (step 411).

FIG. 6(2) shows the results of monitoring carried out by the detection section 104 when the comparative calculation section 116 compared the detection value with the threshold value. FIG. 6(2) shows that the detection value is equivalent to the value (threshold value) for the background in the luminescence reagent solution 103. The results shown in FIG. 6(2) indicate that immersing the nozzle 101 in the luminescence reagent solution 103 removed Adenosine Tri Phosphate remaining in the nozzle 101.

The pressure control section 112 caused the sample solution 503 in the nozzle 101 to be ejected into the luminescence reagent solution 103. The detection section 104 measured luminescence caused by the sample solution 503 and the luminescence reagent solution 103 (step 412).

FIG. 6(3) shows measurement results obtained when 5 atto mol Adenosine Tri Phosphate was added to the luminescence reagent solution 103. An arrow in FIG. 6(3) shows the point in time when the 5 atto mol Adenosine Tri Phosphate in the nozzle 101 was ejected into the luminescence reagent solution 103. FIG. 6(3) indicates that the luminescence occurring when the 5 atto mol Adenosine Tri Phosphate was added to the luminescence reagent solution 103 had an intensity equivalent to that of the luminescence monitored during the washing process (when the nozzle 101 was immersed in the luminescence reagent solution 103 as shown in FIG. 6(1)).

The control section 114 stored the detection value in the storage section 115 as a measurement value. After the storage of the measurement value, to remove Adenosine Tri Phosphate remaining in the nozzle 101, the arm section 113 immersed the nozzle 101 in the luminescence reagent solution 103 as shown in FIG. 5(7). The pressure control section 112 then caused a luminescence reagent solution 505 to be sucked into the nozzle 101 as shown in FIG. 5(8) and caused the luminescence reagent solution 505 in the nozzle 101 to be ejected into the reaction container 106 as shown in FIG. 5(9) (step 413). The detection section 104 monitored luminescence occurring during this process (step 414).

FIG. 6(4) shows the results of monitoring, by the detection section 104, of luminescence occurring when the luminescence reagent solution 505 was sucked into and discharged through the nozzle 101. An arrow in FIG. 6(4) shows the point in time when the luminescence reagent solution 505 in the nozzle 101 was ejected. FIG. 6(4) also shows how Adenosine Tri Phosphate remaining in the nozzle 101 is decomposed as the luminescence attenuates.

The control section 114 stores the monitoring value in the storage section 115 as a detection value. The comparative calculation section 116 compared the stored detection value with the threshold value stored in the storage section 115 (step 415). When the detection value was equivalent to the threshold value, washing of the nozzle 101 was ended (step 416).

The washing and measurement processes shown in FIG. 4 were repeated from step 401 to step 416 to add 1, 2, 10, 50, 100, and 1,000 atto mol Adenosine Tri Phosphate to the luminescence reagent solution 103 so that the detection section 104 measured the resultant luminescence. FIG. 6(5) shows the results of the measurement of the 2 atto mol Adenosine Tri Phosphate. FIG. 6(6) shows the results of the measurement of the 1 atto mol Adenosine Tri Phosphate. Arrows in FIGS. 6(5) and 6(6) show the point in time when the ATP solution in the nozzle 101 was ejected into the luminescence reagent solution 103. As shown in FIGS. 6(1) and 6(4), showing the results of the washing process, if Adenosine Tri Phosphate remains in the nozzle, the luminescence intensity varies or is biased depending on the quantity of Adenosine Tri Phosphate. In this case, the luminescence occurring when the sample solution 105 is added to the luminescence reagent solution 103 contains the luminescence of the Adenosine Tri Phosphate remaining in the nozzle and may thus involve a significant error. However, as shown in FIGS. 6(5) and 6(6), the intensity of the luminescence varies between the addition of the 2 atto mol Adenosine Tri Phosphate and the addition of the 1 atto mol Adenosine Tri Phosphate. This allows the difference in the number of moles to be detected, that is, the 2 atto mol and the 1 atto mol can be distinguishably detected.

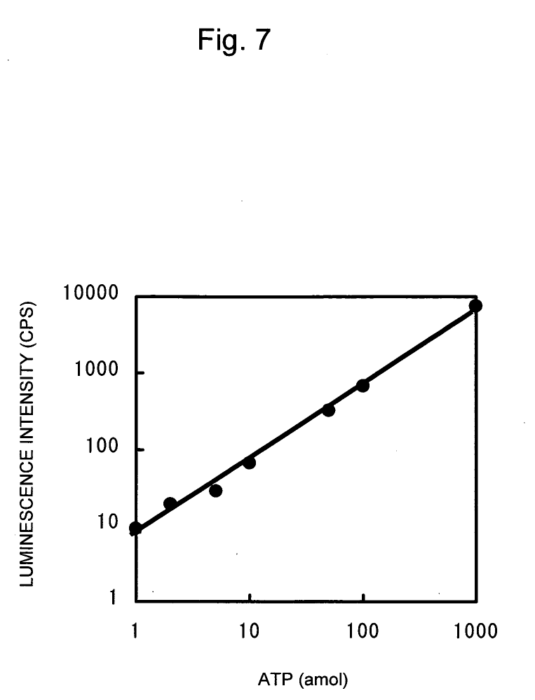

FIG. 7 shows the results of the subtraction of the value for the background in the luminescence reagent solution 103 from the luminescence measurement values for the 1, 2, 5, 10, 50, 100, and 1,000 atto mol Adenosine Tri Phosphate. In FIG. 7, the axis of ordinate indicates the luminescence intensity (CPS). The axis of abscissa indicates the number of moles (atto mol) in the Adenosine Tri Phosphate. The relationship between the luminescence intensity and the number of moles in FIG. 7 exhibits linearity. This indicates that the method according to the present invention can quantify 1 atto mol Adenosine Tri Phosphate and thus has a high quantification capability. Therefore, even with the nozzle 101 (FIG. 6(1) and 6(4)) with Adenosine Tri Phosphate remaining therein, 1 atto mol ATP can be quantitatively measured by using the washing apparatus, washing method, and luminescence measuring method according to the present invention to remove the Adenosine Tri Phosphate remaining in the nozzle.

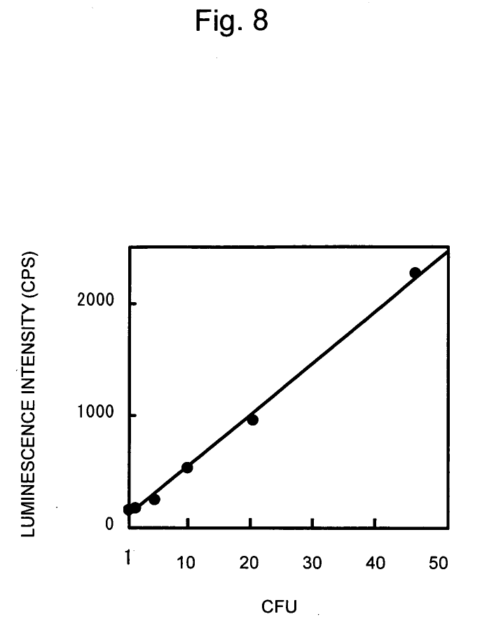

FIG. 8 shows the results of the subtraction of the value for the background in the luminescence reagent solution 103 from the luminescence measurement values for the 1, 2, 5, 10, 20, and 45 CFU. In FIG. 8, the axis of ordinate indicates the luminescence intensity (CPS), and the axis of abscissa indicates the CFU. FIG. 8 shows that the relationship between the luminescence intensity and the CFU exhibits linearity. This indicates that the method according to the present invention can quantify 1 CFU and thus has a high quantification capability. Therefore, even with the nozzle (FIG. 6(1) and 6(4)) with about 5 mol Adenosine Tri Phosphate remaining therein, 1 CFU can be quantitatively measured by using the washing apparatus, washing method, and luminescence measuring method according to the present invention.

The results described above indicate that the luminescence measurement of the sample solution 105 can be accurately and sensitively achieved by removing viable bacteria adhering to the nozzle 101 and Adenosine Tri Phosphate remaining in the nozzle 101. The present invention comprises the detection section 104, which can monitor the washing process. Moreover, the present invention detects luminescence resulting from the decomposition of Adenosine Tri Phosphate. This makes it possible to immediately shift to measurements once the luminescence disappears, that is, once the decomposition of the Adenosine Tri Phosphate ends. Consequently, the time required for the washing process can be reduced.

The present invention enables the microbial count to be accurately and quickly measured. The present invention is thus useful for fields such as clinical medicine sites, food factories, and basic research sites which require sterility and biological cleanliness.

What is claimed is:

1. A luminescence measuring apparatus comprising:
a sample container accommodating a sample solution;
a luminescence reagent vessel accommodating a luminescence reagent solution for detection of the sample solution;
a reaction container in which the sample solution and the luminescence reagent solution are allowed to react chemically;
a detection section detecting luminescence in the reaction container;
a nozzle sucking and ejecting the sample solution;
an arm section controlling operation of the nozzle; and
a pressure control section controlling pressure on the nozzle,
wherein before and after a measuring step of ejecting the sample solution sucked into the nozzle, into the reaction container for luminescence detection, the arm section introduces the nozzle into the reaction container filled with the luminescence reagent solution in order to decompose and wash out a luminescence substrate remaining on a surface of the nozzle.

2. The luminescence measuring apparatus according to claim 1, further comprising a lysys container accommodating a lysys solution, wherein the arm section introduces the nozzle into the lysys solution in order to lyse viable bacteria adhering to the nozzle surface.

3. The luminescence measuring apparatus according to claim 1, wherein the pressure control section controls pressure before and after the sample solution is sucked into the nozzle so that gas or a liquid not mixing with the sample solution is sucked into the nozzle.

4. The luminescence measuring apparatus according to claim 1, further comprising a storage section storing a detection value detected by the detection section and a comparative calculation section comparing the detection value stored in the storage section with a threshold value.

5. The luminescence measuring apparatus according to claim 1, further comprising a control section switching between the washing step and the measurement step on the basis of a result of comparison of a threshold value with a detection value for a background detected during the washing step and/or lysys step and stored in the storage section, the comparison being carried out by the comparative calculation section.

6. A luminescence measuring method comprising executing:
before and after a measurement step of sucking a sample into a nozzle, ejecting the sample solution through the nozzle into a reaction container filled with a luminescence reagent solution, and detecting luminescence in the reaction container,
a washing step of introducing the nozzle into which the sample solution has been sucked, into the reaction container filled with the luminescence reagent solution and decomposing a luminescence substrate remaining on a surface of the nozzle.

7. The method according to claim 6, further comprising a step of, before the washing step, immersing the nozzle into which the sample solution has been sucked, in a lysys solution to lyse viable bacteria adhering to the nozzle surface.

8. The method according to claim 6, further comprising a step of monitoring luminescence detected during the washing step and/or lysys step.

9. The method according to claim 6, further comprising a step of, before and after sucking the sample solution into the nozzle, sucking gas or a liquid not mixing with the sample solution into the nozzle.

10. The method according to claim 6, wherein a detection value for a background detected during the washing step and/or lysys step is compared with a threshold value, and on the basis of a result of the comparison, the washing step and the measurement step are switched.
